(19) United States
(12) Patent Application Publication
(10) Pub. No.: US 2011/0208238 A1
(43) Pub. Date: Aug. 25, 2011

(54) APPLICATOR DELIVERY TIP EXTENSION

(75) Inventor: Jeffrey Michael Hoffman, Acworth, GA (US)

(73) Assignee: CRYOLIFE, INC., Kennesaw, GA (US)

(21) Appl. No.: 13/033,456

(22) Filed: Feb. 23, 2011

Related U.S. Application Data
(60) Provisional application No. 61/307,372, filed on Feb. 23, 2010.
(60) Provisional application No. 61/329,717, filed on Apr. 30, 2010.

(51) Int. Cl. A61B 17/03; B29C 47/02

(52) U.S. Cl. 606/213; 264/255

(57) ABSTRACT

A delivery tip extension communicates an adhesive material from a fluid applicator tip to a site within a patient. The delivery tip extension has an elongated shaft with a proximal end portion, a central body portion, and a tapered distal end portion. The central body portion has a length between about 5 cm and about 45 cm. A lumen is defined within and extends along the elongated shaft from a proximal opening to a distal opening. The elongated shaft is relatively more rigid along the central body portion than along the distal end portion to facilitate driving the elongated shaft through the patient with reduced trauma to the patient by the distal end portion. The lumen is sized and shaped along the proximal end portion for securely receiving the fluid applicator tip inserted through the proximal opening such that a dispensing outlet on the fluid applicator tip is in fluid communication with the lumen. The delivery tip extension is sterilized or sterilizable.

### CROSS-REFERENCES TO RELATED APPLICATIONS

This application claims priority to U.S. Provisional Application No. 61/329,717, filed Apr. 30, 2010, and U.S. Provisional Application No. 61/307,372, filed Feb. 23, 2010, which are incorporated herein by reference.

### TECHNICAL FIELD

The present invention relates generally to the field of biomaterial delivery systems, and more particularly to tip extensions for biomaterial delivery systems.

### BACKGROUND

Fluid biomaterials, such as adhesive substances, are frequently used in surgeries and other medical procedures, for example in soft tissue repairs or as a vascular suture sealant. These materials may be applied by the physician with a syringe or other fluid applicator, such as those described in U.S. Pat. Nos. 4,538,920 and 4,359,049; however, some tissue sites may not be readily accessible with conventional applicators. For example, the site may not be reachable at all or only with great difficulty in an open surgical procedure. In a laparoscopic procedure, delivery may not be possible with a conventional applicator due to site location or applicator limitations. In still other cases, the physician may be unable to navigate through or around soft tissue without damaging the surrounding tissue or organs with a conventional applicator. Often, large incisions are necessary to accommodate such applicator limitations. Large incisions are undesirable.

It would be desirable to provide improved systems for targeted delivery of biomaterials to remote sites within a patient that allow for precise navigation through the body and reduce the risk of damaging surrounding tissue or organs while also decreasing the size of the incision site required. It would also be desirable for the delivery system to provide rapid transport of the biomaterial between the fluid applicator tip and the application site, especially where the fluid to be delivered is a quick-setting adhesive.

### SUMMARY

A delivery tip extension is provided for communicating an adhesive material from a fluid applicator tip to a site within a patient. The delivery tip extension has an elongated shaft with a proximal end portion, a central body portion having a length between 5 cm and 45 cm, and a tapered distal end portion. A lumen is defined within and extends along the elongated shaft from a proximal opening on the proximal end portion to a distal opening on the distal end portion. The elongated shaft is relatively more rigid along the central body portion than along the distal end portion to facilitate driving the elongated shaft through the patient with reduced trauma to the patient by the distal end portion. The lumen is sized and shaped along the proximal end portion for securely receiving the fluid applicator tip inserted through the proximal opening such that a dispensing outlet on the fluid applicator tip is in fluid communication with the lumen. The delivery tip extension is sterilized or sterilizable.

In embodiments, the lumen may have a reduced cross-sectional area along the central body and distal end portions relative to the cross-sectional area along the proximal end portion. The reduced cross-sectional area may promote rapid transport of adhesive from the fluid applicator tip to the distal opening. The delivery tip extension also may include a distal end covering positioned about the distal end portion of the elongated shaft. The distal end covering may include a relatively softer material than the elongated shaft, the relatively softer material further reducing trauma to the patient by the distal end portion. For example, the elongated shaft may include a polyurethane material, and the distal end covering may include a polyether block amide material. The delivery tip extension also may include a proximal end connector positioned about the proximal end portion of the elongated shaft. In some embodiments, the elongated shaft has an outer diameter of between about 2 mm and about 8 mm along the central body portion, and the lumen has a diameter of between about 0.5 mm and about 5.0 mm along the central body and distal end portions. For example, the elongated shaft may have an outer diameter of between about 4 mm and about 6 mm along the central body portion, and the lumen may have a diameter of between about 0.8 mm and about 1.6 mm along the central body and distal end portions. The elongated shaft may have a length of about 10 cm, about 27 cm, or about 35 cm. The proximal end portion may include a textured inner surface for frictional engagement with the fluid applicator tip. The elongated shaft may include a polymer, such as a polyurethane.

In another aspect, a sterile kit is provided for delivering an adhesive material to a site within a patient. The kit includes a syringe apparatus and at least one delivery tip extension. The syringe apparatus has at least one chamber and a fluid applicator tip. The at least one chamber houses a fluid therein, the fluid comprising an adhesive material or at least one precursor therefor. The fluid applicator tip is in fluid communication with the fluid in the chamber, and the fluid applicator tip includes a dispensing outlet. The delivery tip extension is connectable to the fluid applicator tip of the syringe apparatus for dispensing the adhesive. The delivery tip extension has an elongated shaft with a proximal end portion, a central body portion having a length between 5 cm and 45 cm, and a tapered distal end portion. A lumen is defined within and extends along the elongated shaft from a proximal opening on the proximal end portion to a distal opening on the distal end portion. The elongated shaft is relatively more rigid along the central body portion than along the distal end portion to facilitate driving the elongated shaft through the patient with reduced trauma to the patient by the distal end portion. The lumen is sized and shaped along the proximal end portion for securely receiving the fluid applicator tip inserted through the proximal opening such that a dispensing outlet on the fluid applicator tip is in fluid communication with the lumen.

In embodiments, the syringe apparatus may include a first chamber and a second chamber. The first chamber may house a first fluid that comprises a solution including albumin, and the second chamber may house a second fluid that includes an aldehyde for crosslinking the albumin. The syringe apparatus may include a plunger for driving the first and second fluids from the first and second chambers into the fluid applicator tip. The fluid applicator tip may include a static mixer operable to mix the first and second fluids to form the adhesive.

In still another aspect, a method of delivering an adhesive to a tissue site within a patent is provided. The method includes flowing an adhesive from a syringe apparatus through a delivery tip extension to a tissue site in a patient. The delivery tip extension has an elongated shaft with a proximal end portion, a central body portion having a length between 5 cm and 45 cm, and a tapered distal end portion. A lumen is defined within and extends along the elongated shaft from a proximal opening on the proximal end portion to a distal opening on the distal end portion. The elongated shaft is relatively more rigid along the central body portion than along the distal end portion to facilitate driving the elongated shaft through the patient with reduced trauma to the patient by the distal end portion. The lumen is sized and shaped along the proximal end portion for securely receiving the fluid applicator tip inserted through the proximal opening such that a dispensing outlet on the fluid applicator tip is in fluid communication with the lumen.

In embodiments, the delivery tip extension may deliver the adhesive laparoscopically. The delivery tip extension may be inserted through a trocar to deliver the adhesive to the tissue site.

In yet another aspect, a method of making a delivery tip extension is provided. The method includes molding an elongated shaft having a lumen extending therethrough and a length between 5 cm and 45 cm. The elongated shaft includes a proximal end portion, a central body portion, and a tapered distal end portion. A distal end covering is overmolded about the distal end portion of the elongated shaft. The distal end covering comprises a relatively softer material than the elongated shaft. In embodiments, the delivery tip extension may be sterilized.

### BRIEF DESCRIPTION OF THE DRAWINGS

FIG. 1 is an exploded perspective view of a system for delivering a biomaterial to a tissue site in a patient in accordance with one or more embodiments of the present invention.

FIG. 2 is a plan view of one embodiment of a delivery tip extension device.

FIG. 3 is a plan, cross-sectional view of the embodiment of a delivery tip extension device shown in FIG. 2.

FIG. 4 is a plan view of another embodiment of a delivery tip extension device.

FIG. 5 is a plan, cross-sectional view of the embodiment of the delivery tip extension device shown in FIG. 4.

### DETAILED DESCRIPTION

The present application will now be described more fully hereinafter with reference to the accompanying drawings, in which several embodiments of the application are shown. Like numbers refer to like elements throughout the drawings.

Described below are embodiments of systems and methods for delivering a fluid to a location within a patient. The patient may be a human or other mammal, for example. The systems and methods generally employ a fluid dispensing apparatus, such as a syringe, and a delivery tip extension. The fluid dispensing apparatus may dispense a fluid, such as an adhesive, for use within the body of a patient. The delivery tip extension may communicate the fluid from the fluid dispensing apparatus to a location within the patient that is remote from the fluid dispensing apparatus. Thus, the delivery tip extension extends the reach of the fluid dispensing apparatus, so that fluid within the fluid dispensing apparatus can be delivered to a site within a patient that would not be reachable using the fluid dispensing apparatus alone.

In particular embodiments, the delivery tip extension is configured on its proximal end to receive fluid from the fluid delivery apparatus, on its distal end to release the fluid into the patient, and along its length to deliver the fluid between its proximal and distal ends relatively quickly. A lumen may extend along the length of the delivery tip extension for this purpose, and the lumen may be relatively narrow in width. The narrow width of the lumen facilitates quick delivery of the fluid through the length of the delivery tip extension. Thus, when the fluid dispensing apparatus is positioned outside of the patient and the delivery tip extension extends into the patient with its proximal end is in communication with the fluid dispensing apparatus and its distal end is in communication with the intended delivery site, fluid dispensed from the fluid delivery apparatus may quickly travel through the delivery tip extension to reach the intended delivery site. Rapid delivery may be useful in certain surgical contexts, such as embodiments in which the fluid dispensing apparatus dispenses a surgical glue that begins hardening or setting within or shortly upon exiting the fluid delivery apparatus, as described in further detail below.

In particular embodiments, the fluid dispensing apparatus includes at least one chamber and a dispensing tip in communication with the chamber. The chamber may house the fluid, or a component thereof, and the dispensing tip may dispense the fluid from the chamber. The delivery tip extension may secure onto the dispensing tip to receive the fluid therefrom. Thus, when the fluid dispensing apparatus is operated to dispense the fluid from the dispensing tip, the fluid may flow directly into the dispensing tip extension, along its length, and to the intended delivery site.

In particular embodiments, the fluid dispensing apparatus houses at least two fluid components for forming a biocompatible adhesive. For example, the fluid components may be housed in separate chambers, each of which may be in fluid communication with the dispensing tip. The chambers may be operatively associated with a plunger, or any other suitable mechanism for driving the constituent components from the chambers into the dispensing tip. The dispensing tip may include a static mixer or other suitable mechanism for combining the constituent components to react to form an adhesive. The driving force of the plunger may be sufficient to drive the fluid components and resulting adhesive through both the dispensing tip and the delivery tip extension.

In some embodiments, the delivery tip extension includes an elongated shaft and a lumen defined within the elongated shaft between its proximal and distal ends. The elongated shaft may be configured along its proximal end to receive and secure to the fluid dispensing apparatus, and the elongated shaft may be configured along its distal end for controlled dispensing of the biomaterial. For example, the distal end may be tapered.

The elongated shaft may be rigid enough to facilitate navigation through the body tissues. The distal end may be soft to reduce damage to surrounding tissue and organs, and yet the distal end may be stiff enough to ensure the delivery tip extension can navigate through tissues or structures that occlude the target site. The stiffness of the distal end may be sufficient to ensure the end is responsive to direction and torque applied at the proximal end and is less affected by sideways pressure from surrounding tissues/structures. The narrow width of the lumen may provide rapid transport of the fluid along the shaft, which may be useful in cases in which the fluid is an adhesive that begins cross-linking or hardening upon mixing or upon exiting the dispensing tip.

The delivery tip extension may facilitate precise delivery of a fluid or other biomaterial to a delivery site within a patient, even in cases in which the delivery site otherwise is not easily accessible or is inaccessible. The delivery tip extension may be used in an open surgical procedure, a laparoscopic procedure, a video-assisted thoracic surgery, in conjunction with a trocar, or any combination thereof. Examples of open surgeries in which the delivery tip extension may be used include general surgery such as liver resection or pancreatic resection; cardiac surgery such as valve replacement or aneurysm repair; pulmonary surgery such as lung resection, bullectomy, blebectomy, or lung volume reduction; urologic surgery such as partial nephrectomy or prostatectomy; or neurosurgery such as tumor resection or pituitary gland removal. Examples of laparoscopic or video-assisted thoracic surgeries in which the delivery tip extension may be used include general surgery such as liver resection or pancreatic resection; pulmonary surgery such as lung resection, bullectomy, blebectomy, or lung volume reduction; or urologic surgery such as lap partial nephrectomy or lap prostatectomy.

The delivery tip extension may facilitate precise delivery of an adhesive or other fluid to a depth below the surface of the skin that would not be reachable using a standard tip alone, which may be desirable in certain applications. One example is surgery on an obese patient, whose organs may be farther from the surface of the skin. Another example is surgery on an occluded area of the body, such as the back of the heart or the underside of another organ. An additional example is a surgery wherein a portion of the body of the patient is inflated, such as a laparoscopic surgery or other procedure in which the abdominal cavity of the patient is insufflated with carbon dioxide causing the abdominal cavity to enlarge. A trocar, which is often used in such surgeries, is essentially a port through which instruments are introduced into the abdomen. The trocar is often associated with a one-way valve, which prevents carbon dioxide from escaping through the port. Yet another example is a video-assisted thoracic surgery wherein the size of the thoracic cavity necessitates reaching a depth below the surface of the skin or a surgery wherein resected tissue is located closer to the medial line of the body and farther from the skin surface. In these and in other cases, the target tissue may be a distance below the skin surface from which the adhesive or other fluid is deployed, and the delivery tip extension may facilitate accessing these locations effectively.

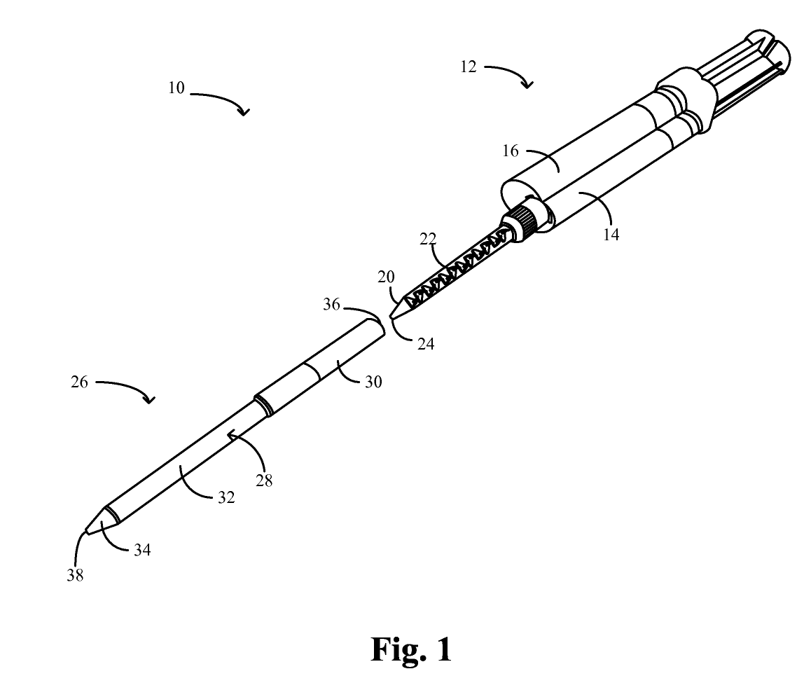

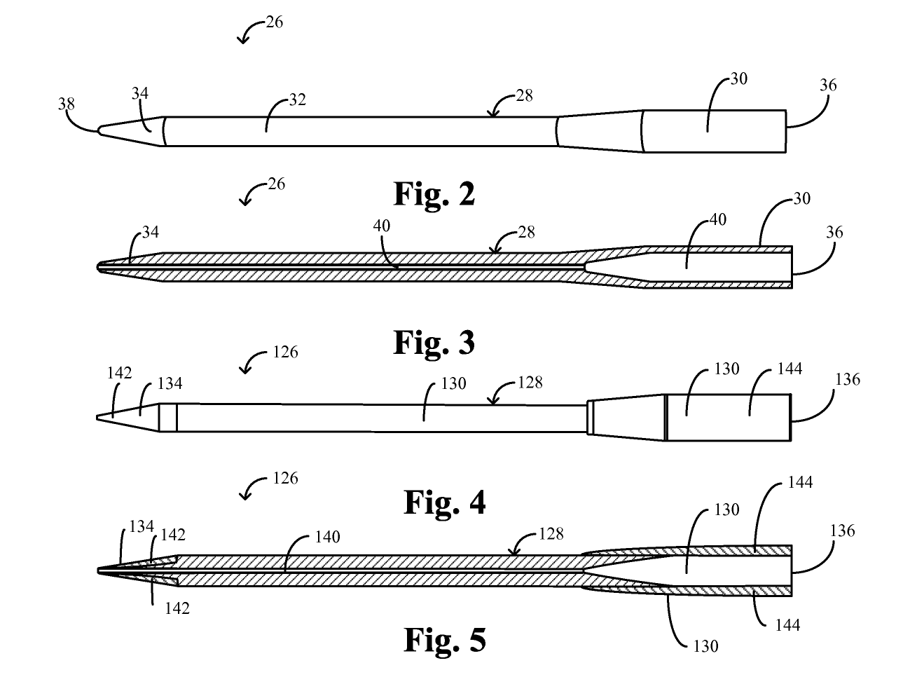

FIG. 1 illustrates an embodiment of a system 10 for delivering a fluid, such as an adhesive, to a location within the body of a patient. The system includes a syringe apparatus 12 and a delivery tip extension 26. The syringe apparatus 12 is configured to dispense a fluid, and the delivery tip extension 26 is configured to deliver the dispensed fluid to a location remote from the syringe apparatus 12. The delivery tip extension 26 is shown in greater detail in FIGS. 2 and 3, which are plan and cross-sectional views.

In the illustrated embodiment, the syringe apparatus 12 includes a first chamber 14, a second chamber 16, and a fluid applicator tip 20 in fluid communication with fluid material contained in the first and second chambers 14, 16. The fluid applicator tip 20 also has a dispensing outlet 24 for dispensing the fluid material. It should be noted that the syringe apparatus 12 may include one chamber or more than two chambers in other embodiments, and that the chambers may have a number or orientations including, including concentric, serial, or side-by-side alignment.

In some embodiments, the fluid material dispensed by the syringe apparatus 12 comprises an adhesive material, such as a bioadhesive material for use within the human body. Each chamber may house an adhesive or an adhesive presursor (e.g., a prepolymer and a crosslinker). The precursors may include a proteinaceous material, such as human or bovine serum albumin, and an aldehyde, including a di- or poly-aldehyde such as gluteraldehyde, as described in U.S. Pat. No. 5,385,606 and No. 7,621,969. The chambers 14, 16 may house these adhesive precursors that can be combined to form an adhesive. For example, one chamber 14 may house a solution that includes albumin, and the other chamber 16 may house an aldehyde for crosslinking the albumin. One example of such an adhesive is BioGlue® surgical adhesive, available from CryoLife, Inc. of Kennesaw, Ga. Such an adhesive may create a bond in thirty seconds or less. Alternatively, the fluid may be another bioadhesive substance known in the art, a biopolymer for tissue augmentation or embolic therapy, a rapid-gelling composition, or the like.

As shown in FIG. 1, the fluid applicator tip 20 may optionally include a static mixer 22 for mixing the fluid material prior to delivery via the dispensing outlet 24. For example, in a dual chamber syringe apparatus 12 wherein each of the two chambers 14, 16 includes an adhesive precursor, the fluid applicator tip 20 may include a static mixer 22 to combine the two adhesive precursors to form an adhesive immediately prior to delivery via the dispensing outlet 24. The adhesive precursors may be driven from the chambers 14, 16 and into the fluid applicator tip 20 with a plunger that is operatively associated with the chambers 14, 16. Upon mixing, such adhesive precursors may immediately begin cross-linking or hardening. In such embodiments, the adhesive may be delivered to the delivery site before it solidifies using a delivery tip extension 26.

More particularly, a delivery tip extension 26 is provided for communicating an adhesive material from a fluid applicator tip 20 of a syringe apparatus 12 to a site within a patient by extending the fluid delivery length of the syringe apparatus 12. The delivery tip extension 26 includes an elongated shaft 28 and a lumen 40. The elongated shaft has a proximal end portion 30, a central body portion 32, and a tapered distal end portion 34. The lumen 40 is defined within and extends along the elongated shaft 28 from a proximal opening 36 on the proximal end portion 30 to a distal opening 38 on the distal end portion 34. The elongated shaft 28 is advantageously relatively more rigid along the central body portion 32 than along the distal end portion 34 to facilitate driving the elongated shaft 28 through/between tissues in the patient. The elongated shaft 28 is relatively less rigid along the distal end portion 34 than along the central body portion 32 to reduce trauma to the patient by the distal end portion 34. The lumen 40 is sized and shaped along the proximal end portion 30 for securely receiving the fluid applicator tip 20 when inserted through the proximal opening 36 such that the dispensing outlet 24 is in fluid communication with the lumen 40.

The distal end portion 34 is relatively less rigid than the central body portion 32, for example, as a result of the reduced wall thickness due to the tapering of the distal end portion 34. The decreased rigidity at the distal end portion 34 reduces the risk of puncturing or tearing surrounding tissue or organs when the delivery tip extension 26 is guided through the patient. In one embodiment, the delivery tip extension 26 is sterilized or sterilizable for use in medical procedures.

In one embodiment, the elongated shaft 28 is made from one or more USP Class VI compliant, clear resins. For example, the elongated shaft 28 may be made from a single gamma-irradiation stable, biocompatible, USP Class VI polymer. In one embodiment, the polymer is a polyurethane, such as Isoplast® 2510. Isoplast® brand polymers are available from Lubrizol Corp. of Wickliffe, Ohio. In another embodiment, the elongated shaft 28 may be made from multiple biocompatible, USP Class VI polymers and resins that are ionizing radiation stable (e.g., gamma-irradiation or electron beam), chemical stable (e.g., glutaraldehyde, ethylene oxide, hydrogen peroxide or plasma), or heat sterilization stable (e.g., autoclaving). Other suitable polymeric materials may be used. Examples of suitable materials include Isoplast® 2530, Pebax® 7033, or Pebax® 7233. Pebax® brand polymeric materials are available from Arkema, Inc. of Philadelphia, Pa.

The elongated shaft 28 may be made from rigid polymers to facilitate easy steering of the delivery tip extension 26 within the body. In an exemplary embodiment, these rigid polymer resins may comprise polyethylene (e.g., low-, high-, or ultrahigh-density), polypropylene, polyurethane, or liquid-crystalline polymers. These resins may include fillers or modifiers. Fillers or modifiers may comprise glass fibers, glass beads, talc, calcium carbonate, or other suitable materials. In certain embodiments, these polymer resins may comprise Vectra® liquid crystal polymers, Isoplast® 2510, Isoplast® 2530, Pebax® 7033, or Pebax® 7233. Vectra® brand polymeric materials are available from Celanese, Inc., Dallas, Tex. Alternatively, the elongated shaft 28 may be made from soft polymer resins for ease of bending or shaping by the end user.

In one embodiment, the tapered distal end portion 34 is conical to provide pointed adhesive application and easy introduction into surgical ports, as shown in FIG. 1. The distal end portion 34 may also be rounded to further minimize tissue damage. In another embodiment, the tapered distal end portion is flattened and flared to dispense a ribbon of the adhesive over the tissue surface. Alternatively, the distal end portion may have other configurations, including those described in U.S. Pat. No. 7,325,995, which is incorporated by reference herein in its entirety.

In particular embodiments, the proximal end portion 36 is configured to engage the fluid applicator tip 20. The engagement may be by frictional engagement, mating threads, snap-fit connection, or the like. The proximal end portion 36 may form a fluid tight connection with the fluid applicator tip 20. In one case, the proximal end portion 36 may include a textured inner surface to promote frictional engagement with the fluid applicator tip 20. The proximal end portion 36 also may be secured to the fluid applicator tip via one or more suitable mechanical fasteners known in the art. Other methods of forming a sealed connection may be used. In some embodiments, the proximal end portion 36 may include a textured outer surface to aid in gripping. For example, the texture may comprise product or brand name, company name, or size identifiers. Alternatively, the texture may comprise random, ordered, or periodic ridges and valleys or shapes.

In certain embodiments, the distal and proximal end portions 30, 34 are made from transparent or translucent polymer resins. The transparent or translucent polymer resins may allow visualization of the fluid being delivered or the fluid applicator tip 20 or static mixer 22 being inserted through the proximal opening 36.

As shown in FIG. 3, the lumen 40 has a reduced cross-sectional area along the central body and distal end portions 32, 34 relative to the cross-sectional area along the proximal end portion 30, to promote rapid transport of adhesive from the fluid applicator tip 20 to the distal opening 38. While the lumen 40 has a cross-sectional area sized along the proximal end portion 36 to securely receive the fluid applicator tip 20 of the syringe apparatus 12, the lumen 40 has a cross-sectional area sized along the central body and distal end portions 32, 34 to provide rapid delivery along the length of the delivery extension tip 26, such as from the dispensing outlet 24 of the fluid applicator tip 20 to the distal opening 38 of the delivery extension tip 26 for application at the tissue site within the patient. In cases in which the adhesive is one that solidifies quickly, the reduced diameter of the lumen 40 provides almost immediate communication of the adhesive from the fluid applicator tip to the site of application, thereby reducing adhesive hardening within the delivery tip extension 26. Also, in some embodiments, the lumen 40 is smooth and has a low coefficient of friction to further expedite fluid transport along the lumen 40.

In some embodiments, the elongated shaft 28 has an outer diameter of between about 0.1 mm and about 20 mm, and the lumen 40 has a diameter of between about 0.01 mm and about 19 mm along the central body and distal end portions 32, 34. In particular embodiments, the elongated shaft 28 has an outer diameter of between about 1 mm and about 10 mm, and the lumen 40 has a diameter of between about 0.5 mm and about 4.5 mm along the central body and distal end portions 32, 34.

In one embodiment, the elongated shaft 28 is constructed of a clear polyurethane, e.g., Isoplast® 2510 and has an outer diameter between about 2 mm and about 8 mm along the central body portion and the lumen has a diameter of between about 0.5 mm and about 5.0 mm along the central body and distal end portions. In one particular embodiment, the elongated shaft 28 has an outer diameter between about 4 mm and about 6 mm (e.g., about 5 mm) along the central body portion and the lumen has a diameter of between about 0.8 mm and about 1.6 mm (e.g., about 1.2 mm) along the central body and distal end portions. However, in other embodiments the elongated shaft 28 may have a larger outer diameter, such as a diameter that is as much as 20 mm or more, so that the entire delivery tip extension is somewhat stiffer or more rigid. A stiffer or more rigid delivery tip extension may be useful in certain surgical applications.

In one embodiment, the elongated shaft 28 has a length between 5 cm and 45 cm. In exemplary embodiments, the elongated shaft 28 has a length of about 10 cm, about 27 cm, or about 35 cm. Other lengths of the elongated shaft 28 are also envisioned.

FIGS. 4 and 5 illustrate another embodiment of a delivery tip extension 126. In this embodiment, the delivery tip extension 126 includes a distal end covering 142 positioned about the distal end portion 134 of the elongated shaft 128. The distal end covering 142 comprises a relatively softer material than the elongated shaft 128, to further reduce trauma to the patient by the distal end portion 134. In some embodiments, the relatively softer material of the distal end covering 142 is a soft polymer resin, such as a polyether block amide, polyurethane, or polyethylene. In one embodiment, the distal end covering 142 comprises Pebax® 4033 SA01. The delivery tip extension 126 may be formed with a step or indent along the distal end portion 134 so that a smooth surface is formed when the distal end covering 142 is positioned thereabout. For example, the distal end covering 142 may be formed by overmolding.

In some embodiments, the delivery tip extension 126 also includes a proximal end connector 144 positioned about the proximal end portion 130 of the elongated shaft 128, as shown in FIGS. 4 and 5. The proximal end connector 144 may be separately formed from the elongated shaft 128 and attached thereto, such as by overmolding or with an adhesive, so that the proximal end connector 144 can be formed from a different material than the elongated shaft 128. For example, the elongated shaft 128 may be relatively rigid or stiff, so that the elongated shaft can be directed to the area of application, while the proximal end connector 144 may have a certain flexibility or give so that it can stretch about and flexibly mate with the applicator tip. As another example, the elongated shaft 128 may have a relatively lower coefficient of friction so that it can slide into and out of a trocar without excessive frictional engagement, while the proximal end connector 144 may exhibit a higher coefficient of friction. The higher coefficient of friction for the proximal end connector 144 may facilitate grasping the connector to place it on the applicator tip. The higher coefficient of friction also may facilitate retaining the proximal end connector 144 on the applicator tip. These various design parameters may be achieved by forming different portions of the delivery tip extension 126 from different materials, with different diameters, with different wall thicknesses, or any combination thereof. Thus, the elongated shaft 128 may be associated with a separate proximal end connector 144 to form the delivery tip extension 126 in some embodiments.

In another aspect, a system is provided for delivering an adhesive material to a site within a patient. The system includes the syringe apparatus and the delivery tip extension. The syringe apparatus is operably connected to the delivery tip extension by inserting the fluid applicator tip through the proximal opening and securing it within the proximal end portion such that the dispensing outlet is in fluid communication with the lumen.

The system may be used to deliver adhesive, sealant or tissue augmentation compositions to bond, coat, or augment a variety of tissue sites. The adhesive may bond to living tissues, including muscle, skin, connective tissue, nerve tissue, vascular and cardiac tissues, cartilage, bone, and the like, as well as to corresponding cadaver tissues, which may be preserved or otherwise chemically treated. Bonds may also be formed to natural or synthetic materials such as rubber, polyethylene terephthalate, polytetrafluoroethylene, and the like, as well as to metals, enabling the use of these compositions for the attachment of surgical grafts and devices, as well as for wound closure, trauma repair, and the like in the practice of human or veterinary medicine. Non-medical applications of the adhesive delivery system are also envisioned.

In another aspect, a method is provided for delivering an adhesive to a tissue site in a patient. The method includes flowing an adhesive from a syringe apparatus through a delivery tip extension to a tissue site in a patient. In some embodiments, the delivery tip extension delivers the adhesive laparoscopically. In an exemplary embodiment, the delivery tip extension is used in conjunction with a trocar to deliver the adhesive to the tissue site.

In another aspect, a method is provided for making a delivery tip extension. The method includes a first step of molding, extruding, thermoforming, or a combination thereof, an elongated shaft having a lumen extending therethrough and a length between 5 cm and 45 cm. The elongated shaft includes a proximal end portion, a central body portion, and a tapered distal end portion. In some embodiments, the method includes a next step of overmolding a distal end covering about the distal end portion of the elongated shaft, the distal end covering comprising a relatively softer material than the elongated shaft. In some embodiments, the method for making a delivery tip extension also includes a step of overmolding a proximal end connector about the proximal end portion of the elongated shaft. Overmolding distal and proximal end portions on an elongated shaft may facilitate forming different portions of the delivery tip extension from different materials. Thus, the materials of the different portions may be specifically selected based on the desired function of the different portions. Additionally, overmolding may facilitate varying the diameter of the delivery tip extension along its length with improved quality control and less waste.

In particular embodiments, the elongated shaft is extruded from rigid polyurethane (e.g., Isoplast® 2530) and the distal and/or proximal end coverings are made from soft polyether block amide (e.g., Pebax® 4033 SA01) which is overmolded onto the elongated shaft.

In some embodiments, the delivery tip extension components may be formed separately and then assembled or combined together. In other embodiments, one or more preparation steps may be included between the component formation and combination steps. The preparation step may be of a physical or mechanical nature, for example shaving, thermoforming, crimping, cutting, flaring, or other steps known in the art. Alternatively, the preparation step may be of a chemical nature, for example solvent application, degreasing, priming, or other steps known in the art. The combination step may be accomplished by one or more steps that include gluing the components or surfaces together, bonding by chemical means, bonding by physical techniques (e.g., heat, laser, ultrasound, radio frequency, or friction), thermoforming or overmolding, or other suitable means.

The delivery tip extension may be packaged for shipping and storage. It may be sterilized once manufactured/assembled, either before or after packaging. Sterilization may be achieved either by ionizing radiation sterilization, chemical sterilization, or heat sterilization. Suitable examples includes gamma or electron beam ionizing radiation or elthylene oxide (EtO) gas sterilization. Other suitable irradiation processes may be used. As used herein, the term “sterilizable” in reference to the delivery tip extension means that the device is constructed of one or more materials that are stable when subjected to an effective sterilization process.

One or more delivery tip extensions may be packaged together with one or more applicators to provide a kit. In one embodiment, the kit contains different sizes of delivery tip extensions together with an applicator syringe containing BioGlue™ (CryoLife, Inc., Kennesaw, Ga. USA) or another surgical adhesive. In one embodiment, the delivery tip extension is packaged in a double pouch, such as one having an inner pouch and an outer pouch. The delivery tip extension is packaged two tips per pouch and ten pouches per box. The box preferably includes an Instruction For Use (IFU) insert, in the appropriate languages.

Publications cited herein and the materials for which they are cited are specifically incorporated by reference. Modifications and variations of the methods and devices described herein will be obvious to those skilled in the art from the foregoing detailed description. Such modifications and variations are intended to come within the scope of the appended claims.

We claim:

1. A delivery tip extension for communicating an adhesive material from a fluid applicator tip to a site within a patient, comprising:
an elongated shaft comprising a proximal end portion, a central body portion having a length between 5 cm and 45 cm, and a tapered distal end portion; and
a lumen defined within and extending along the elongated shaft from a proximal opening on the proximal end portion to a distal opening on the distal end portion;
wherein the elongated shaft is relatively more rigid along the central body portion than along the distal end portion to facilitate driving the elongated shaft through the patient with reduced trauma to the patient by the distal end portion,
wherein the lumen is sized and shaped along the proximal end portion for securely receiving the fluid applicator tip inserted through the proximal opening such that a dispensing outlet on the fluid applicator tip is in fluid communication with the lumen, and
wherein the delivery tip extension is sterilized or sterilizable.

2. The delivery tip extension of claim 1, wherein the lumen has a reduced cross-sectional area along the central body and distal end portions relative to the cross-sectional area along the proximal end portion, the reduced cross-sectional area promoting rapid transport of adhesive from the fluid applicator tip to the distal opening.

3. The delivery tip extension of claim 1, further comprising a distal end covering positioned about the distal end portion of the elongated shaft, the distal end covering comprising a relatively softer material than the elongated shaft, the relatively softer material further reducing trauma to the patient by the distal end portion.

4. The delivery tip extension of claim 3, wherein:
the elongated shaft comprises a polyurethane material; and
the distal end covering comprises a polyether block amide material.

5. The delivery tip extension of claim 1, further comprising a proximal end connector defining the proximal end portion of the elongated shaft, the proximal end connector comprising a relatively softer material than the elongated shaft.

6. The delivery tip extension of claim 1, wherein:
the elongated shaft has an outer diameter of between about 2 mm and about 8 mm along the central body portion; and
the lumen has a diameter of between about 0.5 mm and about 5.0 mm along the central body and distal end portions.

7. The delivery tip extension of claim 4, wherein:
the elongated shaft has an outer diameter of between about 4 mm and about 6 mm along the central body portion; and
the lumen has a diameter of between about 0.8 mm and about 1.6 mm along the central body and distal end portions.

8. The delivery tip extension of claim 1, wherein the elongated shaft has a length of about 10 cm, about 27 cm, or about 35 cm.

9. The delivery tip extension of claim 1, wherein the proximal end portion comprises a textured inner surface for frictional engagement with the fluid applicator tip.

10. The delivery tip extension of claim 1, wherein the elongated shaft comprises a clear polymer.

11. The delivery tip extension of claim 9, wherein the polymer comprises a polyurethane.

12. A sterile kit for delivering an adhesive material to a site within a patient, comprising:
a syringe apparatus comprising:
at least one chamber housing a fluid that comprises an adhesive or at least one precursor therefor;
a fluid applicator tip in fluid communication with each fluid in each chamber, the fluid applicator tip having a dispensing outlet; and
at least one delivery tip extension connectable to the fluid applicator tip of the syringe apparatus for dispensing the adhesive, the delivery tip extension comprising:
an elongated shaft comprising a proximal end portion, a central body portion having a length between 5 cm and 45 cm, and a tapered distal end portion; and
a lumen defined within and extending along the elongated shaft from a proximal opening on the proximal end portion to a distal opening on the distal end portion;
wherein the elongated shaft is relatively more rigid along the central body portion than along the distal end portion to facilitate driving the elongated shaft through the patient with reduced trauma to the patient by the distal end portion, and
wherein the lumen is sized and shaped along the proximal end portion for securely receiving the fluid applicator tip inserted through the proximal opening such that the dispensing outlet is in fluid communication with the lumen.

13. The system of claim 12, wherein the at least one chamber comprises a first chamber and a second chamber.

14. The system of claim 13, wherein:
the first chamber houses a first fluid that comprises a solution including albumin; and
the second chamber houses a second fluid that comprises an aldehyde for crosslinking the albumin.

15. The system of claim 13, wherein the syringe apparatus further comprises a plunger for driving the first and second fluids from the first and second chambers into the fluid applicator tip.

16. The system of claim 14, wherein the fluid applicator tip comprises a static mixer operable to mix the first and second fluids to form the adhesive.

17. The system of claim 12, wherein the fluid applicator tip comprises a static mixer.

18. A method for delivering an adhesive to a tissue site in a patient, comprising:
flowing an adhesive from a syringe apparatus through a delivery tip extension to a tissue site in a patient, the delivery tip extension comprising:
an elongated shaft comprising a proximal end portion, a central body portion having a length between 5 cm and 45 cm, and a tapered distal end portion; and
a lumen defined within and extending along the elongated shaft from a proximal opening on the proximal end portion to a distal opening on the distal end portion;
wherein the elongated shaft is relatively more rigid along the central body portion than along the distal end portion to facilitate driving the elongated shaft through the patient with reduced trauma to the patient by the distal end portion;
wherein the lumen is sized and shaped along the proximal end portion for securely receiving the fluid applicator tip inserted through the proximal opening such that a dispensing outlet on the fluid applicator tip is in fluid communication with the lumen.

19. The method of claim 18, wherein the delivery tip extension delivers the adhesive laparoscopically.

20. A method of making a delivery tip extension, comprising:
molding an elongated shaft having a lumen extending therethrough and a length between 5 cm and 45 cm, the elongated shaft comprising:
a proximal end portion,
a central body portion, and
a tapered distal end portion; and
overmolding a distal end covering about the distal end portion of the elongated shaft, the distal end covering comprising a relatively softer material than the elongated shaft, to form the delivery tip extension.

21. The method of claim 20, further comprising sterilizing the delivery tip extension.
